SEQUENCE LISTING (2 sequences)

SEQ ID NO: 1 - DNA, 1410 bp, Homo sapiens
cggaattagggagatagttggtattaggattaggattgttgtgaagtatagtacggatgctacttgtgcaatgatggtaaaagggtagcttactggttgtcctccgattcaggttagaatgaggaggtctgcggcttggagctcctggggcctaacaaaaagaaacctgccatgctgctcttcctcctctctgcactggtcctactcacacagcccctgggctacctggaagcagaaatgaagacctactcccacagaacaacgcccagtgcttgcaccctggtcatgtgtagctcagtggagagtggcctgcctggtcgcgatggacgggatgggagagagggccctcggggcgagaagggggacccaggtttgccaggagctgcagggcaagcagggatgcctggacaagctggcccagttgggcccaaaggggacaatggctctgttggagaacctggaccaaagggagacactgggccaagtggacctccaggacctcccggtgtgcctggtccagctggaagagaaggtcccctggggaagcaggggaacataggacctcagggcaagccaggcccaaaaggagaagctgggcccaaaggagaagtaggtgccccaggcatgcagggctcggcaggggcaagaggcctcgcaggccctaagggagagcgaggtgtccctggtgagcgtggagtccctggaaacgcaggggcagcagggtctgctggagccatgggtccccagggaagtccaggtgccaggggacccccgggattgaagggggacaaaggcattcctggagacaaaggagcaaagggagaaagtgggcttccagatgttgcttctctgaggcagcaggttgaggccttacagggacaagtacagcacctccaggctgctttctctcagtataagaaagttgagctcttcccaaatggccaaagtgtcggggagaagattttcaagacagcaggctttgtaaaaccatttacggaggcacagctgctgtgcacacaggctggtggacagttggcctctccacgctctgccgctgagaatgccgccttgcaacagctggtcgtagctaagaacgaggctgctttcctgagcatgactgattccaagacagagggcaagttcacctaccccacaggagagtccctggtctattccaactgggccccaggggagcccaacgatgatggcgggtcagaggactgtgtggagatcttcaccaatggcaagtggaatgacagggcttgtggagaaaagcgtcttgtggtctgcgagttctgagccaactggggtgggtggggcagtgcttggcccaggagtttggccagaagtcaaggcttagaccctcatgctgccaatatcctaataaaaaggtgaccatcaaaaaaaaaa

SEQ ID NO: 2 - protein, 374 aa, Homo sapiens
MLLFLLSALVLLTQPLGYLEAEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNAGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDGGSEDCVEIFTNGKWNDRACGEKRLVVCEF

(19) United States
(12) Patent Application Publication
(10) Pub. No.: US 2010/0143920 A1
(43) Pub. Date: Jun. 10, 2010

(54) Surfactant Protein D is a Biomarker for Steroid Responsiveness in Asthma and Chronic Obstructive Pulmonary Disease

(75) Inventors: Reynold A. Panettieri, Jr., Bensalem, PA (US); Michael F. Beers, Wayne, PA (US); Angela F. Haczku, Princeton Junction, NJ (US); Michael W. Sims, Mount Laurel, NJ (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: The Trustees of the University of Pennsylvania

(21) Appl. No.: 12/594,536

(22) PCT Filed: Apr. 2, 2008
(86) PCT No.: PCT/US08/04302

§ 371 Date: Feb. 12, 2010

Related U.S. Application Data
(60) Provisional application No. 60/922,116, filed on Apr. 6, 2007.

(51) Int. Cl. G01N 33/53; C07H 21/04; C07K 17/00; C12Q 1/68

(52) U.S. Cl. 435/6; 536/23.5; 530/402; 530/391.1; 530/391.3; 435/7.1; 435/7.92

(57) ABSTRACT

The present invention relates to the identification of a biomarker, the detection of which is prognostic for individuals who are responsive to inhalation of corticosteroids as a therapy for asthma and COPD.

### BACKGROUND OF THE INVENTION

Chronic obstructive pulmonary disease (COPD) is the fourth leading cause of death in the United States, claiming the lives of 122,283 Americans in 2003 alone. In 2004, 11.4 million adults over the age of 18 were estimated to have COPD; however, over 24 million U.S. adults have evidence of impaired lung function, indicating an under-diagnosis of COPD (American Lung Association, 2006, Trends in Chronic Bronchitis and Emphysema Morbidity and Mortality). The cost to the nation for COPD in 2004 was approximately $37.2 billion dollars, including healthcare expenditures of $20.9 billion in direct health care expenditures, $7.4 billion in indirect morbidity costs, and $8.9 billion in indirect mortality costs (American Lung Association, 2006, Trends in Chronic Bronchitis and Emphysema Morbidity and Mortality).

Smoking is responsible for 90% of COPD in the United States. Although not all cigarette smokers will develop COPD, it is estimated that 15% will. Smokers with COPD have higher death rates than nonsmokers with COPD. They also have more frequent respiratory symptoms (coughing, shortness of breath, etc.) and more deterioration in lung function than non-smokers.

COPD is comprised primarily of two related diseases: chronic bronchitis and emphysema. Chronic bronchitis is the inflammation and eventual scarring of the lining of the bronchial tubes. When the bronchi are inflamed and/or infected, less air is able to flow to and from the lungs and a heavy mucus or phlegm is coughed up. The condition is defined by the presence of a mucus-producing cough most days of the month, three months of a year for two successive years without other underlying disease to explain the cough. Emphysema begins with the destruction of air sacs (alveoli) in the lungs where oxygen from the air is exchanged for carbon dioxide in the blood, due in part, by an abnormal inflammatory response of the lung to noxious particles or gases, chiefly cigarette smoke. The walls of the air sacs are thin and fragile. Damage to the air sacs is irreversible and results in permanent “holes” in the tissues of the lower lungs. As air sacs are destroyed, the lungs are able to transfer less and less oxygen to the bloodstream, causing shortness of breath. The lungs also lose their elasticity, which is important to keep airways open. As a result, the patient experiences great difficulty exhaling. In both chronic bronchitis and emphysema the obstruction is generally permanent and progressive.

The quality of life for a person suffering from COPD diminishes as the disease progresses. At the onset, there is minimal shortness of breath, but people with COPD may eventually require supplemental oxygen and mechanical respiratory assistance. A recent American Lung Association survey revealed that half of all COPD patients (51%) say their condition limits their ability to work. It also limits them in normal physical exertion (70%), household chores (56%), social activities (53%), sleeping (50%) and family activities (46%).

Asthma is another pulmonary disease in which there is obstruction to the flow of air out of the lungs, but unlike chronic bronchitis and emphysema, the obstruction in asthma is usually transient and reversible; between “attacks” of asthma, the flow of air through the airways is usually good.

Some patients with COPD obstruction can be partially reversed by medications that enlarge or dilate the airways (bronchodilators) as in the treatment of asthma. Conversely, some asthmatic patients can develop permanent airway obstruction if chronic inflammation of the airways leads to scarring and narrowing of the airways in a process called lung remodeling. Asthma patients with a fixed component of airway obstruction are also considered to have COPD.

There are four main surfactant proteins known as SP-A, SP-B, SP-C and SP-D. Surfactant Protein D (SP-D), together with SP-A and mannose binding lectin (MBL) is a member of the “collectin” family of structurally related Ca²⁺ dependent lectins that share collagen-like N-terminal tails and globular lectin heads containing C-type carbohydrate recognition domains. Produced in alveolar type II cells and Clara cells, SP-D is a 43-kD monomer which forms a higher order quaternary structure.

SP-D has myriad effects on innate immunity, including binding to and enhancing clearance of a wide variety of pathogens (Bufler et al., 2003, Am. J. Respir. Cell Mol. Biol., 28:249-56; Ferguson et al., 2006, Infect. Immun. 74:7005-9; Hartshorn et al., 2006, J. Immunol. 176:6962-72; LeVine et al., 2004, Am. J. Respir. Cell Mol. Biol. 31:193-9; Ofek et al., 2001, Infect. Immuno. 69:24-33; Vuk-Pavlovic et al., 2001, Am. J. Respir. Cell Mol. Biol. 24:475-84), promoting phagocytosis of apoptotic macrophages (Clark et al., 2003, Ann. N.Y. Acad. Sci. 1010:113-6; Vandivier et al., J. Immunol. 169:3978-86), stimulating chemotaxis of neutrophils (Madan et al., 1997, Infect. Immun. 65:3171-9; Crouch et al., 1995, Am. J. Respir. Cell Mol. Biol. 12:410-5) and macrophages (Crouch, 1998, Am. J. Respir. Cell Mol. Biol. 12:177-201; Tino and Wright, 1999, Am. J. Physiol. 276:L164-74), and inhibiting pro-inflammatory cytokine release by effector cells (Limper et al., 1995, J. Lab. Clin. Med. 126:416-22; Borron et al., 1998, J. Immunol. 161:4599-603; LeVine and Whitsett, 2001, Microbes Infect. 3:161-6; Haczku, 2006, Pharmacol. Ther. 110:14-34). Because of the immunoprotective properties of SP-D, constitutive expression in the proximal and distal airspaces appears essential in order to maintain an immunologically hyporeactive tissue milieu under normal (non-infective) conditions.

In addition to its effects on innate immunity, SP-D also plays an important role in regulating inflammation in the lung. Even in the absence of inflammatory stimuli, SP-D deficient mice display an abnormal pulmonary phenotype characterized by accumulations of lipid-laden macrophages in the aveoli, pulmonary emphysema, perivascular and peribronchial lymphocytic infiltrate, and increased expression of matrix metalloproteases (Atochina et al., 2004, Am. J. Respir. Cell Mol. Biol. 30:271-9; Botas et al., 1998, Proc. Natl. Acad. Sci. U.S.A. 95:11869-74; Fisher et al., 2002, Am. J. Respir. Cell Mol. Biol. 27:24-33; Wert et al., 2000, Proc. Natl. Acad. Sci. U.S.A. 97:5972-7). In addition, these mice are more susceptible to lung injury from a variety of insults including bleomycin, hyperoxia, ozone challenge, allergic sensitization, and viral, bacterial or pneumonocystis infection (LeVine and Whitsett, 2001, Microbes Infect. 3:161-6; Atochina et al., 2004, Am. J. Respir. Cell Mol. Biol. 30:271-9; Casey et al., 2005, Am. J. Respir. Crit. Care Med. 172:869-77; Kierstein et al., 2006, Respir. Res. 7:85; Haczku et al., 2006, J. Immunol. 176:3557-65).

Susceptibility to O₃-induced inflammatory changes varies between different mice strains and is associated with different basal levels of SP-D expression. For example, C57BL/6 mice express high levels of SP-D as well as IL-10 and IL-6 (both anti-inflammatory). In contrast, Balb/c mice release more KC and IL-12p70 and exhibit a greater pulmonary inflammatory response and cell death as a result of O₃ exposure than their C57BL/6 counterparts. Reduced SP-D levels or a lack of SP-D, as in the case of knock out mice, predispose an individual to a severe inflammatory response upon exposure to an allergen or irritant, such as O₃. Indeed, induction of SP-D by prior endotoxin exposure protects animals from O₃ induced cell death and pulmonary inflammation (Bachurski et al., 2001, Am. J. Physiol. Lung Cell Mol. Physiol. 280:L279-85; Li et al., 2000, Inhal. Toxicol. 12:1225-1238). Studies examining the effect of SP-D and other collectins on the production of inflammatory mediators have generated seemingly contradictory data, suggesting both suppression (Borron et al., 1998, J. Immunol. 161:4599-603; Harrod et al., 1999, Am. J. Physiol. 277: L580-8; Kremlev and Phelps, 1994, Am. J. Physiol. 267:L712-9) and induction (Ofek et al., 2001, Infect. Immuno. 69:24-33; Kremlev and Phelps, 1994, Am. J. Physiol. 267:L712-9) of pro-inflammatory cytokine secretion by macrophages.

These opposing effects of SP-D on inflammation are mediated by different portions of the SP-D molecule. The globular heads of SP-D inhibit pro-inflamatory mediator release via binding of SIRPα and resultant inhibition of the p38 MAP kinase pathway. In contrast, the collagenous tail of SP-D, which is shared by the other collectins SP-A and MBL as well as collectin-like C1q, can serve as a ligand for receptors such as calreticulin/CD91 (Vandivier et al., 2002, J. Immunol. 169:3978-86; Malhotra et al., 1992, Eur. J. Immunol. 22:1437-45; Ogden et al., 2001, J. Exp. Med. 194:781-95; McGreal and Gasque, 2002, Biochem. Soc. Trans. 30:1010-4; Vandivier et al., 2002, Chest, 121:89 S; Gardai et al., 2003, Cell 115:13-23), activating the p38 MAP kinase pathway and promoting release of pro-inflammatory mediators by alveolar macrophages. Thus SP-D serves a dual role, both promoting and abrogating the immune response to infections and injury.

SP-D like other collectins, forms higher order structures and is capable of forming multimers with a total mass of several million kDa and a size of about 100 nm (Crouch et al., 1994, J. Biol. Chem. 269:17311-9). In vivo, SP-D is typically present as a dodecamer. The hydrophobic N-terminal collagen (pro-inflammatory) domains are well hidden in the center of the multimeric structures and are bound together by cysteine residues, while the globular heads (anti-inflamatory) are exposed at the periphery. Because of the inhibitory effects of the globular heads of SP-D on the release of pro-inflammatory mediators, the native, multimeric form exhibits predominantly anti-inflammatory properties.

In contrast, monomeric and trimeric forms of SP-D have exposed collagenous tails thereby favoring the pro-inflammatory properties of SP-D. Unmasking the hydrophobic N-terminal collagen domain of SP-D by degradation of multimers into monomers and trimers may initiate or amplify pro-inflamatory macrophage activity. This mechanism serves to efficiently clear pathogens, but it can also have unwanted deleterious effects for inflammation produced by non-infectious insults such as cigarette smoke.

Cigarette smoke exposure results in high levels of free oxygen or nitrogen radicals that can damage the cysteine bonds between the SP-D oligomers, unravel the multimeric structure, expose the collagen receptor ligand and convert the immunosuppressive function of SP-D into a pro-inflammatory one. SP-D appears to be particularly susceptible to the presence of nitric oxide (Gow wt al., 2004, Methods Mol. Biol. 279:167-72; Gow et al., 2004, Am J. Physiol. Lung Cell Mol. Physiol. 287:L262-8; Ischiropoulos and Gow, 2005, Toxicology 208:299-303).

Nitric Oxide (NO), a free radical product of mammalian cell metabolism plays diverse and important roles in the regulation of cell function (Gow and Ischiropoulos, 2001, J. Cell Physiol. 187:277-282; Lane et al., 2001, Sci. STKE 2001 86:RE1). While NO can also undergo redox transfer with other biomolecules such as lipids and DNA, proteins are a major target of NO reactivity. Formation of SP-D multimers depends on the cysteine residues at positions 15 and 20 within the hydrophobic tail domain that hold the multimeric SP-D structure together by covalently linked disulfide bonds (Crouch et al., 1994, J. Biol. Chem. 269:17311-9; Brown-Augsburger et al., 1996, J. Biol. Chem. 271:18912-9; Brown-Augsburger et al., 1996, J. Biol. Chem. 271:13724-30). At physiological concentrations of NO, oxidation to N₂O₃ is facilitated by micellar catalysis, which is mediated by the hydrophobic phase of target proteins. Thus, unlike other protein modifications such as phosphorylation or acetylation, N₂O₃ forms inside protein-hydrophobic cores and nitrosylates the protein interior. A target protein such as the multimeric form of SP-D is thus a catalyst of its own nitrosylation (Nedospasov et al., 2000, Proc. Natl. Acad. Sci. U.S.A. 97:13543-8). Disruption of disulfide bonds on cysteine residues within the hydrophobic domain by oxidative or nitrative damage results in degradation of the higher order multimeric structure and invokes the proinflammatory effects of SP-D.

Expression levels of SP-D are affected by pulmonary injury and inflammation. For example, SP-D mRNA and immunoreactive protein are induced within several hours of treatment of bacterial endotoxin (McIntosh et al., 1996, Am. J. Respir. Cell Mol. Biol. 15:509-19), Pseudomonas aeruginosa (Jain-Vora et al., 1998, Infect. Immunol. 66:4229-36), exposure to hyperoxia (Aderibigbe et al., 1999, Am. J. Respir. Cell Mol. Biol. 20:219-27), and chronic infection with Pneumocystis carinii (Atochina et al., 2000, Am. J. Physiol. Lung Cell Mol. Physiol. 278:L599-609) in experimental rodent models. In a rodent model of allergic inflammation, SP-D expression was also elevated (Hackzu et al., 2006, J. Immunol. 176:3557-65).

Two reports have demonstrated that SP-D levels in bronchoalveolar lavage fluid (BALF) obtained from humans are reduced as a direct result of smoking (Honda et al., 2007, Chest 109:1006-1009; Betsukyaku et al., 2004, Eur. J. Respir. 24:964-970). For example, the SP-D concentration in BALF recovered from smokers was significantly lower (0.5±0.1 μg/ml; p less than 0.05) than that in BALF recovered from non-smokers (1.3±0.2 μg/ml) with no significant difference in total protein (Honda et al., 2007, Chest 109:1006-1009).

Glucocorticoid is the most effective anti-inflammatory agent available for use in treating asthma. Allergic airway inflammation is characterized by increased pulmonary expression of pro-inflammatory cytokines and adhesion molecules, including SP-D. In a murine model of allergic inflammation, dexamethasone was able to dose-dependently suppress the elevated BALF levels of SP-D as measured by two-dimensional gels and western blots in addition to substantially reducing the total cell number and eosinophils recovered in BALF from asthmatic mice (Zhao et al., 2007, Int. Arch. Allergy Immunol. 142:219-229).

None of the existing medications for COPD has been shown to modify the long-term decline in lung function that is the hallmark of this disease. Therefore, the goal of pharmacotherapy for COPD is to provide relief of symptoms and prevent complications and/or progression of the disease with minimal side effects. Bronchodilator medications are central to the symptomatic management of COPD. They can be inhaled as aerosol sprays or taken orally. Additional treatment includes antibiotics, oxygen therapy, and systemic glucocorticosteroids. However, chronic treatment with systemic steroids involves significant risk of serious side effects and can be used for no longer than two weeks; therefore, these are reserved mostly for acute exacerbations of COPD. The efficacy of inhaled glucocorticosteroids is largely unknown, however observational studies suggest short-term benefit. A clear problem in the art, however, is that, as in asthma, not every patient responds to inhaled glucocorticoid therapy. At present, there is no biomarker available to identify patients responsive to inhaled glucocorticoid therapy; there are only indirect measures of steroid responsiveness including spirometry (a direct measure of airflow obstruction) and the use of exhaled nitric oxide as a measure of airway inflammation. The present invention fulfills this unmet need.

### SUMMARY OF THE INVENTION

The invention includes a method of identifying an individual having lung disease that is responsive to treatment for the lung disease. The method comprises measuring the level of a biomarker in a body sample obtained from the individual, wherein when the level of the biomarker in the sample is greater than the level of the same biomarker in an otherwise identical sample obtained from the same individual prior to commencement of treatment, this is an indication that the individual is responsive to the treatment.

In this and other embodiments of the invention, the individual is a mammal selected from the group consisting of a mouse, a rat, a non-human primate, and a human. Preferably, the mammal is a human.

In this and other embodiments of the invention, the individual has COPD or asthma.

In still other embodiments, the biomarker is a surfactant protein, more preferably surfactant protein D.

In other embodiments, the treatment is administration of a corticosteroid, preferably, inhalation of a corticosteroid.

In still other embodiments, the body sample is selected from the group consisting of a tissue, a cell and a bodily fluid, wherein the bodily fluid comprises pulmonary sputum and/or bronchoalveolar lavage fluid.

In yet another aspect of the invention, the method of measuring the biomarker comprises an immunoassay for assessing the level of the biomarker in a sample, wherein the immunoassay is selected from the group consisting of Western blot, ELISA, immunoprecipitation, immunohistochemistry, immunofluorescence, radioimmunoassay, dot blotting, and FACS.

In still a further aspect of the invention, the method of measuring the biomarker comprises a nucleic acid assay for assessing the level of a nucleic acid encoding the biomarker in the sample wherein the nucleic assay is selected from the group consisting of a Northern blot, Southern blot, in situ hybridization, a PCR assay, and an RT-PCR assay.

The invention also includes a method of assessing the efficacy of a treatment for lung disease in an individual. The method comprises obtaining a first sample from the individual having the lung disease and measuring the level of SP-D in the first sample, obtaining a second sample from the same individual at a time subsequent to obtaining the first sample, wherein the time is subsequent to the onset of treatment, and measuring the level of SP-D in the second sample, wherein when the level of SP-D is higher in the second sample than the level of SP-D in the first sample, the treatment is efficacious.

In still another aspect of the invention, the measuring of the biomarker comprises an immunoassay for assessing the level of said biomarker in the sample. In a further aspect of the invention, the measuring of the biomarker comprises a nucleic acid assay for assessing the level of a nucleic acid encoding the biomarker in the sample.

The present invention also includes a composition comprising a plurality of oligonucleotides attached to a substrate surface. Each of the oligonucleotides is a nucleic acid encoding a biomarker or a fragment thereof, or is complementary to the biomarker or a fragment thereof, wherein the biomarker is surfactant protein D.

In one embodiment of the invention, the substrate surface is a membrane, a chip, a bead, a microsphere or a microchip.

The invention further includes a composition comprising a plurality of peptides attached to a substrate surface, wherein each of the peptides is a biomarker or a fragment thereof, wherein the biomarker is surfactant protein D.

In another embodiment, the substrate surface is a membrane, a chip, a bead, a microsphere or a microchip.

The invention also includes a composition comprising a plurality of antibodies attached to a substrate surface wherein the antibody specifically binds a biomarker or a fragment thereof, wherein the biomarker is surfactant protein D.

In another aspect, the antibody comprises a detectable label, wherein said detectable label is selected from the group consisting of a radioactive, a fluorescent, a biological, and an enzymatic label.

The invention further includes a kit comprising a composition for measuring the level of a biomarker in a body sample. The composition comprises at least one antibody that specifically binds the biomarker or a fragment thereof, the kit comprising instructional material for the use thereof.

In one embodiment, the composition comprises at least one antibody that specifically binds a biomarker or a fragment thereof, wherein the biomarker is surfactant protein D.

In another embodiment of the invention, at least one of the antibodies is bound to a substrate surface. In a preferred embodiment, the antibody comprises a detectable label, wherein said detectable label is selected from the group consisting of a radioactive, a fluorescent, a biological, and an enzymatic label.

The invention also includes a kit comprising a composition for measuring the level of a biomarker in a body sample. The composition comprises at least one nucleic acid, wherein the nucleic acid encodes the biomarker or a fragment thereof, or is complementary to the biomarker or a fragment thereof, the kit further comprising an instructional material for the use thereof.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A and FIG. 1B, is a series of images illustrating the structure of surfactant protein D (SP-D). FIG. 1A illustrates that the collectins are composed of structurally similar C-terminal carbohydrate recognition domains (CRDs) attached to a neck and an N-terminal collagenous region. FIG. 1B illustrates the quaternary structure of collectins formed by higher order oligomers bound through the N-terminally located cysteines, forming a “tetrameric cross.”

FIG. 2 is a chart illustrating the subject characteristics of the individuals included in the data set.

FIG. 3 is a chart depicting SP-D levels in BALF according to smoking and disease status. P value shown on the graph is for the overall trend in SP-D levels by a nonparametric test for trend. *p=0.01 for the difference between healthy former smokers and former smokers with COPD. ** p=0.45 for the difference between healthy current smokers and current smokers with COPD.

FIG. 4, comprising FIG. 4A through FIG. 4D, is a series of images depicting dexamethasone upregulation of SP-D but not SP-A by type II cells in vitro. FIG. 4A is an image depicting Western Blot analysis for cells isolated from lungs of adult rat and cultured for a four day period either with or without dexamethasone (10 nM). FIG. 4B is a graph expressing the results of A as % of the day 0 value. *p=0.037 (n=3). FIG. 4C is an image depicting Northern blot analysis of SP-D and SP-A mRNA expression performed on total RNA (10 μg) extracted from type II cells either cultured with or without dexamethasone. FIG. 4D is a graph depicting the quantification of the results from FIG. 4C using densitometry to quantify the intensity of the band. Hybridization signals were normalized to 28s RNA probe labeled with [γ³²P] ATP. mRNA content is expressed as % of the “day 0” value. *p<0.041 Mean±SEM was calculated after deriving the average of the results from three independent experiments.

FIG. 5, comprising FIG. 5A and FIG. 5B, is a series of images depicting the sequence for SP-D. FIG. 5A depicts the nucleotide sequence of human SP-D (Accession No.: X65018) and FIG. 5B depicts the protein sequence of human SP-D (Accession No.: P35247).

### DETAILED DESCRIPTION OF THE INVENTION

The present invention provides compositions and methods for detecting elevated levels of a biomarker in an individual, wherein levels of the biomarker are elevated as a result of treatment of the individual for lung disease. The compositions and methods of the present invention include biomarkers whose levels are altered in response to treatment, wherein the biomarker is preferably surfactant protein D, and wherein the treatment administered to an individual comprises any treatment that is either known or heretofor unknown, that ameliorates the symptoms of lung disease. Examples of such lung disease include, but are not limited to, COPD and asthma. In the present invention, a preferred treatment is the administration of a corticosteroid to an individual having lung disease, preferably wherein the corticosteroid is inhaled.

The invention permits the evaluation of the efficiency of treatment of an individual with lung disease, the evaluation of the potential efficiency of treatment of an individual with lung disease, and an assessment of the individual's lung disease state. In one aspect, the method of the invention comprises collecting a body sample from an individual and measuring the level of SP-D in the body sample as an indicator of the disease state of the individual. In another aspect, the method of the invention also includes collecting a body sample from an individual and measuring the level of SP-D in the body sample as an indicator of that individual's responsiveness to treatment for lung disease. In yet another aspect, the method of the invention includes collecting a first body sample from an individual and measuring the level of SP-D in said first body sample, then collecting a subsequent body sample from the same individual and measuring the level of SP-D in the subsequent body sample. When the level of SP-D is elevated in the subsequent body sample relative to the level of SP-D measured in the first body sample, treatment of the individual for lung disease is efficacious.

In still another aspect, the method of the invention embodies collecting sequential body samples from an individual with lung disease throughout the duration of treatment, measuring the level of SP-D in each of said body samples collected, and comparing the level of SP-D measured in each body sample to levels of SP-D measured in body samples collected previously or subsequently. When the level of SP-D measured in said body samples rises relative to levels of SP-D measured in previous body samples or maintains an acceptable level throughout the duration of treatment, said treatment for lung disease is efficacious. In one embodiment of the present invention, the body sample collected comprises pulmonary sputum or BALF. In preferred embodiments, multiplexed ELISAs are used to detect SP-D levels in a body sample.

The biomarkers of the invention that are most useful in the methods of the invention include proteins whose levels increase or genes that encode said proteins whose expression increases as a result of treatment. Biomarkers of particular interest include surfactant protein D.

The level of any biomarker is assessed in a sample obtained from the individual at the protein or nucleic acid level. In some embodiments of the invention immunohistochemistry techniques are provided that utilize antibodies to detect over or under-expression of biomarkers in biological samples. Expression of biomarkers can also be detected by nucleic acid-based techniques, including, but not limited to, hybridization techniques and RT-PCR. Kits comprising reagents for practicing the methods of the invention are further provided.

### DEFINITIONS

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used in this specification and the appended claims, the singular forms “a”, “an”, and “the” include plural referents unless the content clearly dictates otherwise. Thus, for example, reference to “a pulmonary surfactant” includes a combination of two or more pulmonary surfactants, and the like.

An “amino acid” as used herein is meant to include both natural and synthetic amino acids, and both D and L amino acids. “Standard amino acid” means any of the twenty L-amino acids commonly found in naturally occurring peptides. “Nonstandard amino acid residues” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides, and particularly at the carboxy- or amino-terminus, can be modified by methylation, amidation, acetylation or substitution with other chemical groups which can change a peptide's circulating half life without adversely affecting activity of the peptide. Additionally, a disulfide linkage may be present or absent in the peptides.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). As used herein, a “neutralizing antibody” is an immunoglobulin molecule that binds to and blocks the biological activity of the antigen.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The phrase “body sample” as used herein, is intended any sample comprising a cell, a tissue, or a bodily fluid in which expression of a biomarker can be detected. Examples of such body samples include but are not limited to pulmonary sputum and bronchoalveolar lavage fluid, blood, lymph biopsies, and smears. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art.

The phrase “chronic obstructive pulmonary disease” or “COPD”, as used herein, means a process characterized by the presence of chronic bronchitis or emphysema that may lead to the development of airway obstruction, both reversible airway obstruction and irreversible airway obstruction.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anti-codon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “DNA” as used herein is defined as deoxyribonucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “fragment,” as applied to a protein or peptide, refers to a subsequence of a larger protein or peptide. A “fragment” of a protein or peptide can be at least about 20 amino acids in length; for example at least about 50 amino acids in length; at least about 100 amino acids in length, at least about 200 amino acids in length, at least about 300 amino acids in length, and at least about 400 amino acids in length (and any integer value in between).

The term “heterologous” as used herein is defined as DNA or RNA sequences or proteins that are derived from the different species.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. By way of example, the DNA sequences 3′ATTGCC5′ and 3′TATGGC are 50% homologous.

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

By the term “specifically binds,” as used herein, is meant an antibody which recognizes and binds a biomarker or fragment thereof, but does not substantially recognize or bind other molecules in a sample.

The term “therapeutic” as used herein means a treatment and/or prophylaxis. A therapeutic effect is obtained by suppression, remission, or eradication of a disease state associated with liver disease.

The term “treatment” as used within the context of the present invention is meant to include therapeutic treatment as well as prophylactic, or suppressive measures for the disease or disorder. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

### DESCRIPTION

The present invention discloses that cigarette smoke not only specifically decreases the levels of SP-D present in BALF, but can also alter the molecular structure of SP-D to favor its inflammatory function. The present invention includes the discovery that the level of SP-D in a body sample collected from an individual is diagnostic for disease status in the individual. The invention also includes the discovery that elevated levels of SP-D in response to a treatment for lung disease are prognostic for improved clinical outcome. It has been discovered in the present invention that inhaled corticosteroid causes an elevation of SP-D levels measured in pulmonary sputum and BALF. Thus, for the first time, there is disclosed a specific biomarker wherein elevated levels of the biomarker in response to treatment are predictive of an individual responsive to that treatment and are prognostic of an improved clinical outcome.

The present invention provides compositions and methods for directly assessing an individual's responsiveness to inhaled corticosteroids, particularly when that individual has been diagnosed with asthma and/or COPD.

In certain embodiments, the methods comprise the detection of a specific biomarker whose expression levels can be detected in the fluid obtained from an individual during bronchoalveolar lavage or from pulmonary sputum. The biomarker is preferably surfactant protein D (SP-D). That is, detection of the level of the biomarker of the current invention distinguishes between a patient who is responsive or has responded to therapy, particularly to inhaled corticosteroids, and one who does not or who has not responded to therapy. Further, the present invention enables the practitioner to make an assessment as to whether or not inhaled corticosteroids or other therapy for asthma and/or COPD are likely to be of therapeutic benefit to a particular individual. The method for detecting an individual likely to be responsive to inhaled corticosteriod treatment involves the detection of SP-D in a body sample, preferably BALF. In particular embodiments, antibodies and immunohistochemistry techniques are used to detect expression of the biomarker of interest. In other embodiments, biomarker levels are detected by detecting nucleic acid levels. Kits for practicing the methods of the invention are further provided.

### Biomarkers

The biomarker to be measured in the methods of the invention includes genes and proteins, and variants and fragments thereof, that exhibit a change in level as a result of treatment of the individual for COPD and/or asthma. The preferred treatment is inhaled corticosteroids. Such biomarkers include DNA comprising the entire or partial sequence of the nucleic acid sequence encoding the biomarker, or the complement of such a sequence. Biomarker nucleic acids useful in the invention should be considered to include both DNA and RNA comprising the entire or partial sequence of any of the nucleic acid sequences of interest. A biomarker protein should be considered to comprise the entire or partial amino acid sequence of any of the biomarker proteins or polypeptides.

A “biomarker” is any gene, protein, or metabolite whose level of expression in a tissue, cell or bodily fluid is altered compared to that of an untreated individual, cell, tissue, or biological fluid. Preferred biomarkers to be measured in the methods of the invention selectively respond to treatment modalities for COPD and/or asthma, preferably the inhalation of corticosteroids.

By “selectively respond to inhalation of corticosteriods” it is intended that the level of the biomarker of interest is elevated in response to inhaled corticosteriods and is associated with an individual who will respond to treatment with inhaled corticosteroids with clinical improvement. Measuring the levels of the biomarker in the methods of the invention permits differentiation between samples collected from a healthy non-smoking individual, a healthy smoking individual, a healthy former smoker, a non-smoker with COPD, a former smoker with COPD and a current smoker with COPD. Further, by measuring the levels of the biomarker in the method of the invention, a sample obtained from an individual would directly assess that individual's disease progression.

The present invention also provides for analogs of polypeptides which comprise a biomarker protein. Analogs may differ from naturally occurring proteins or polypeptides by conservative amino acid sequence differences or by modifications which do not affect sequence, or by both. For example, conservative amino acid changes may be made, which although they alter the primary sequence of the protein or polypeptide, do not normally alter its function (e.g., secretion and capable of blocking virus infection). Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.
   Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

The present invention should also be construed to encompass “mutants,” “derivatives,” and “variants” of the biomarker proteins of the invention (or of the DNA encoding the same) which mutants, derivatives and variants are altered in one or more amino acids (or, when referring to the nucleotide sequence encoding the same, are altered in one or more base pairs) such that the resulting peptide (or DNA) is not identical to the sequences recited herein, but has the same biological property as SP-D disclosed herein, in that the proteins have biological/biochemical properties. A biological property of the polypeptides of the present invention should be construed but not be limited to include, the ability to mediate pulmonary immune response to infection and injury.

Further, the invention should be construed to include naturally occurring variants or recombinantly derived mutants of biomarker protein sequences, which render the polypeptide encoded thereby either more, less, or just as biologically active as wild type biomarker proteins.

The measurement of biomarkers in the methods of the invention distinguishes between individuals who do respond or have responded to treatment, particularly inhalation of corticosteroids, and individuals who either have not received treatment or who have not responded to treatment.

Currently available techniques, such as spirometry, only test pulmonary function and are not able to predict an individual's responsiveness to treatment. The present invention provides a clinician with a new, direct method for establishing the likelihood of treatment efficacy as well as monitoring treatment efficacy, in an individual by measuring the levels of a specific biomarker.

In some embodiments, the methods for assessing steroid responsiveness are performed as a reflex to a preexisting clinical situation, such as an individual diagnosed with COPD or asthma. In other aspects of the invention, the methods are performed as a primary screening test for individuals at risk of developing COPD.

### Detection

In particular embodiments, the diagnostic methods of the invention comprise collecting a sample from a patient, contacting the sample with at least one antibody specific for a biomarker of interest, and detecting antibody binding thereto both pre- and post exposure to inhaled corticosteriods. Samples that contain a elevated levels of SP-D after exposure to ICS identify an individual that can be characterized as steroid responsive and likely to benefit from a course of treatment with ICS.

Any methods available in the art for identification or detection of the biomarkers are encompassed herein. The change of a biomarker's expression level in the invention can be detected at a nucleic acid level or a protein level. In order to determine expression of the biomarker, levels of the biomarker are measured in the body sample to be examined and compared with a corresponding body sample that originates from the same individual prior to treatment with ICS. In another embodiment of the invention, clinical status of the individual is determined by measuring the levels of the biomarker in the body sample to be examined and comparing with an average value obtained from more than one not-at-risk individuals. In still another embodiment of the invention, level of the biomarker is determined by measuring levels of the biomarker in the body sample to be examined and comparing with levels of biomarker obtained from a body sample obtained from the same individual at a different time as an indicator of disease progression.

Methods for detecting biomarker of the invention comprise any method that determines the quantity or the presence of the biomarker either at the nucleic acid or protein level. Such methods are well known in the art and include but are not limited to western blots, northern blots, southern blots, ELISA, immunoprecipitation, immunofluorescence, flow cytometry, immunocytochemistry, nucleic acid hybridization techniques, nucleic acid reverse transcription methods, and nucleic acid amplification methods. In particular embodiments, dysregulation of a biomarker is detected on a protein level using, for example, antibodies that are directed against specific biomarker proteins. These antibodies can be used in various methods such as Western blot, ELISA, immunoprecipitation, or immunocytochemistry techniques.

The invention should not be limited to any one method of protein or nucleic acid detection method recited herein, but rather should encompass all known or heretofor unknown methods of detection as are, or become, known in the art.

In one embodiment, antibodies specific for biomarker proteins are used to detect levels of a biomarker protein in a body sample. The method comprises obtaining a body sample from a patient, contacting the body sample with at least one antibody directed to a biomarker whose expression is selectively altered in response to ICS. One of skill in the art will recognize that the immunocytochemistry method described herein below is performed manually or in an automated fashion.

When the antibody used in the methods of the invention is a polyclonal antibody (IgG), the antibody is generated by inoculating a suitable animal with a biomarker protein, peptide or a fragment thereof. Antibodies produced in the inoculated animal which specifically bind the biomarker protein are then isolated from fluid obtained from the animal. Biomarker antibodies may be generated in this manner in several non-human mammals such as, but not limited to goat, sheep, horse, rabbit, and donkey. Methods for generating polyclonal antibodies are well known in the art and are described, for example in Harlow, et al. (1998, In: Antibodies, A Laboratory Manual, Cold Spring Harbor, N.Y.). These methods are not repeated herein as they are commonly used in the art of antibody technology.

When the antibody used in the methods of the invention is a monoclonal antibody, the antibody is generated using any well known monoclonal antibody preparation procedures such as those described, for example, in Harlow et al. (supra) and in Tuszynski et al. (1988, Blood, 72:109-115). Given that these methods are well known in the art, they are not replicated herein. Generally, monoclonal antibodies directed against a desired antigen are generated from mice immunized with the antigen using standard procedures as referenced herein. Monoclonal antibodies directed against full length or peptide fragments of biomarker may be prepared using the techniques described in Harlow, et al. (1998, In: Antibodies, A Laboratory Manual, Cold Spring Harbor, N.Y.).

Samples may need to be modified in order to render the biomarker antigens accessible to antibody binding. In a particular aspect of the immunocytochemistry methods, slides are transferred to a pretreatment buffer, for example phosphate buffered saline containing Triton-X. Incubating the sample in the pretreatment buffer rapidly disrupts the lipid bilayer of the cells and renders the antigens (i.e., biomarker proteins) more accessible for antibody binding. The pretreatment buffer may comprise a polymer, a detergent, or a nonionic or anionic surfactant such as, for example, an ethyloxylated anionic or nonionic surfactant, an alkanoate or an alkoxylate or even blends of these surfactants or even the use of a bile salt. The pretreatment buffers of the invention are used in methods for making antigens more accessible for antibody binding in an immunoassay, such as, for example, an immunocytochemistry method or an immunohistochemistry method.

Any method for making antigens more accessible for antibody binding may be used in the practice of the invention, including antigen retrieval methods known in the art. See, for example, Bibbo, 2002, Acta. Cytol. 46:25 29; Saqi, 2003, Diagn. Cytopathol. 27:365 370; Bibbo, 2003, Anal. Quant. Cytol. Histol. 25:8 11. In some embodiments, antigen retrieval comprises storing the slides in 95% ethanol for at least 24 hours, immersing the slides one time in Target Retrieval Solution pH 6.0 (DAKO S1699)/dH2O bath preheated to 95° C., and placing the slides in a steamer for 25 minutes.

Following pretreatment or antigen retrieval to increase antigen accessibility, samples are blocked using an appropriate blocking agent, e.g., a peroxidase blocking reagent such as hydrogen peroxide. In some embodiments, the samples are blocked using a protein blocking reagent to prevent non-specific binding of the antibody. The protein blocking reagent may comprise, for example, purified casein, serum or solution of milk proteins. An antibody directed to a biomarker of interest is then incubated with the sample.

Techniques for detecting antibody binding are well known in the art. Antibody binding to a biomarker of interest may be detected through the use of chemical reagents that generate a detectable signal that corresponds to the level of antibody binding and, accordingly, to the level of biomarker protein expression. In one of the preferred immunocytochemistry methods of the invention, antibody binding is detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Enzymes of particular interest include horseradish peroxidase (HRP) and alkaline phosphatase (AP). Commercial antibody detection systems, such as, for example the Dako Envision+ system (Dako North America, Inc., Carpinteria, Calif.) and Mach 3 system (Biocare Medical, Walnut Creek, Calif.), may be used to practice the present invention.

In one particular immunocytochemistry method of the invention, antibody binding to a biomarker is detected through the use of an HRP-labeled polymer that is conjugated to a secondary antibody. Antibody binding can also be detected through the use of a mouse probe reagent, which binds to mouse monoclonal antibodies, and a polymer conjugated to HRP, which binds to the mouse probe reagent. Slides are stained for antibody binding using the chromogen 3,3-diaminobenzidine (DAB) and then counterstained with hematoxylin and, optionally, a bluing agent such as ammonium hydroxide or TBS/Tween-20. In some aspects of the invention, slides are reviewed microscopically by a cytotechnologist and/or a pathologist to assess cell staining (i.e., biomarker overexpression). Alternatively, samples may be reviewed via automated microscopy or by personnel with the assistance of computer software that facilitates the identification of positive staining cells.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

In regard to detection of antibody staining in the immunocytochemistry methods of the invention, there also exist in the art video-microscopy and software methods for the quantitative determination of an amount of multiple molecular species (e.g., biomarker proteins) in a biological sample, wherein each molecular species present is indicated by a representative dye marker having a specific color. Such methods are also known in the art as colorimetric analysis methods. In these methods, video-microscopy is used to provide an image of the biological sample after it has been stained to visually indicate the presence of a particular biomarker of interest. Some of these methods, such as those disclosed in U.S. patent application Ser. No. 09/957,446 and U.S. patent application Ser. No. 10/057,729 to Marcelpoil, incorporated herein by reference, disclose the use of an imaging system and associated software to determine the relative amounts of each molecular species present based on the presence of representative color dye markers as indicated by those color dye markers' optical density or transmittance value, respectively, as determined by an imaging system and associated software. These techniques provide quantitative determinations of the relative amounts of each molecular species in a stained biological sample using a single video image that is “deconstructed” into its component color parts.

The antibodies used to practice the invention are selected to have high specificity for the biomarker proteins of interest. Methods for making antibodies and for selecting appropriate antibodies are known in the art. See, for example, Celis, J. E. ed. (in press) Cell Biology & Laboratory Handbook, 3rd edition (Academic Press, New York), which is herein incorporated in its entirety by reference. In some embodiments, commercial antibodies directed to specific biomarker proteins may be used to practice the invention. The antibodies of the invention may be selected on the basis of desirable staining of cytological, rather than histological, samples. That is, in particular embodiments the antibodies are selected with the end sample type (i.e., cytology preparations or ELISA) in mind and for binding specificity.

One of skill in the art will recognize that optimization of antibody titer and detection chemistry is needed to maximize the signal to noise ratio for a particular antibody. Antibody concentrations that maximize specific binding to the biomarkers of the invention and minimize non-specific binding (or “background”) will be determined in reference to the type of biological sample being tested. In particular embodiments, appropriate antibody titers for use cytology preparations are determined by initially testing various antibody dilutions on formalin-fixed paraffin-embedded normal tissue samples. Optimal antibody concentrations and detection chemistry conditions are first determined for formalin-fixed paraffin-embedded tissue samples. The design of assays to optimize antibody titer and detection conditions is standard and well within the routine capabilities of those of ordinary skill in the art. After the optimal conditions for fixed tissue samples are determined, each antibody is then used in cytology preparations under the same conditions. Some antibodies require additional optimization to reduce background staining and/or to increase specificity and sensitivity of staining in the cytology samples.

Furthermore, one of skill in the art will recognize that the concentration of a particular antibody used to practice the methods of the invention will vary depending on such factors as time for binding, level of specificity of the antibody for the biomarker protein, and method of body sample preparation. Moreover, when multiple antibodies are used, the required concentration may be affected by the order in which the antibodies are applied to the sample, i.e., simultaneously as a cocktail or sequentially as individual antibody reagents. Furthermore, the detection chemistry used to visualize antibody binding to a biomarker of interest must also be optimized to produce the desired signal to noise ratio.

As noted, it is contemplated that the biomarkers of the invention will find utility as immunogens, e.g., in immunohistochemistry and in ELISA assays. One evident utility of the encoded antigens and corresponding antibodies is in immunoassays for the detection of biomarker proteins, as needed in diagnosis and prognostic monitoring.

### Immunoassays

Immunoassays, in their simplest and most direct sense, are binding assays. Certain preferred immunoassays are the various types of enzyme linked immunosorbent assays (ELISA) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, antibodies binding to the biomarker proteins of the invention are immobilized onto a selected surface exhibiting protein affinity, such as a well in a polystyrene microtiter plate. Then, a test composition suspected of containing the biomarker antigen, such as a clinical sample, is added to the wells. After binding and washing to remove non-specifically bound immunecomplexes, the bound antibody may be detected. Detection is generally achieved by the addition of a second antibody specific for the target protein, that is linked to a detectable label. This type of ELISA is a simple “sandwich ELISA”. Detection may also be achieved by the addition of a second antibody, followed by the addition of a third antibody that has binding affinity for the second antibody, with the third antibody being linked to a detectable label.

In another exemplary ELISA, the samples suspected of containing the biomarker antigen are immobilized onto the well surface and then contacted with the antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Another ELISA in which the proteins or peptides are immobilized, involves the use of antibody competition in the detection. In this ELISA, labeled antibodies are added to the wells, allowed to bind to the biomarker protein, and detected by means of their label. The amount of marker antigen in an unknown sample is then determined by mixing the sample with the labeled antibodies before or during incubation with coated wells. The presence of marker antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immunecomplexes. These are described as follows:

In coating a plate with either antigen or antibody, the wells of the plate are incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating of nonspecific adsorption sites on the immobilizing surface reduces the background caused by nonspecific binding of antisera to the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control and/or clinical or biological sample to be tested under conditions effective to allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

“Under conditions effective to allow immunecomplex (antigen/antibody) formation” means that the conditions preferably include diluting the antigens and antibodies with solutions such as, but not limited to, BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours, at temperatures preferably on the order of 25° to 27° C., or may be overnight at about 4° C.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this label is an enzyme that generates a color or other detectable signal upon incubating with an appropriate chromogenic or other substrate. Thus, for example, the first or second immunecomplex can be detected with a urease, glucose oxidase, alkaline phosphatase or hydrogen peroxidase-conjugated antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation is then achieved by measuring the degree of color generation, e.g., using a visible spectra spectrophotometer.

### Nucleic Acid-Based Techniques

In other embodiments, the expression of a biomarker of interest is detected at the nucleic acid level. Nucleic acid-based techniques for assessing expression are well known in the art and include, for example, determining the level of biomarker mRNA in a body sample. Many expression detection methods use isolated RNA. Any RNA isolation technique that does not select against the isolation of mRNA can be utilized for the purification of RNA from body samples (see, e.g., Ausubel, ed., 1999, Current Protocols in Molecular Biology (John Wiley & Sons, New York). Additionally, large numbers of tissue samples can readily be processed using techniques well known to those of skill in the art, such as, for example, the single-step RNA isolation process of Chomczynski, 1989, U.S. Pat. No. 4,843,155).

The term “probe” refers to any molecule that is capable of selectively binding to a specifically intended target biomolecule, for example, a nucleotide transcript or a protein encoded by or corresponding to a biomarker. Probes can be synthesized by one of skill in the art, or derived from appropriate biological preparations. Probes may be specifically designed to be labeled with a detectable label. Examples of molecules that can be used as probes include, but are not limited to, RNA, DNA, proteins, antibodies, and organic molecules.

Isolated mRNA as a biomarker can be detected in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a biomarker of the present invention. Hybridization of an mRNA with the probe indicates that the biomarker in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array (Santa Clara, Calif.). A skilled artisan can readily adapt known mRNA detection methods for use in detecting the level of mRNA encoded by the biomarkers of the present invention.

An alternative method for determining the level of biomarker mRNA in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189 193), self sustained sequence replication (Guatelli, 1990, Proc. Natl. Acad. Sci. USA, 87:1874 1878), transcriptional amplification system (Kwoh, 1989, Proc. Natl. Acad. Sci. USA, 86:1173 1177), Q-Beta Replicase (Lizardi, 1988, Bio/Technology, 6:1197), rolling circle replication (Lizardi, U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan® System). Such methods typically use pairs of oligonucleotide primers that are specific for the biomarker of interest. Methods for designing oligonucleotide primers specific for a known sequence are well known in the art.

Biomarker expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of biomarker expression may also comprise using nucleic acid probes in solution.

### Microarray

In one embodiment of the invention, microarrays are used to detect biomarker expression in biological samples. Microarrays are particularly well suited for this purpose because of the reproducibility between trials. DNA microarrays provide one method for the simultaneous measurement of the expression levels of large numbers of genes or a large number of oligonucletide probes directed to different parts of a molecule of interest. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261, incorporated herein by reference in its entirety for all purposes. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. Arrays may be packaged in such a manner as to allow for diagnostics or other manipulation of an all-inclusive device. See, for example, U.S. Pat. Nos. 5,856,174 and 5,922,591 herein incorporated by reference.

Nucleic acids which code for the biomarkers can be placed in an array on a substrate, such as on a chip (e.g., DNA chip or microchips). These arrays also can be placed on other substrates, such as microtiter plates, beads or microspheres. Methods of linking nucleic acids to suitable substrates and the substrates themselves are described, for example, in U.S. Pat. Nos. 5,981,956; 5,922,591; 5,994,068 (Gene Logic's Flow-thru ChipO Probe ArraysO); U.S. Pat. Nos. 5,858,659, 5,753,439; 5,837,860 and the FlowMetrix technology (e.g., microspheres) of Luminex (U.S. Pat. Nos. 5,981,180 and 5,736,330).

There are two preferred methods to make a nucleic acid array. One is to synthesize the specific oligonucleotide sequences directly onto the solid-phase in the desired pattern (Southern, 1994, Nucl. Acids Res., 22: 1368-73; Maskos, 1992, Nucl. Acids Res., 20: 1679-84; Pease, 1994, Proc. Natl. Acad. Sci., 91: 5022-6; and U.S. Pat. No. 5,837,860) and the other is to presynthesize the oligonucleotides in an automated DNA synthesizer and then attach the oligonucleotides onto the solid-phase support at specific locations (Lamture, 1994, Nucl. Acids Res., 22: 2121; Smith, 1994, Nucl. Acids Res., 22: 5456 64. In the first method, the efficiency of the coupling step of each base affects the quality and integrity of the nucleic acid molecule array.

A second, more preferred method for nucleic acid array synthesis utilizes an automated DNA synthesizer for DNA synthesis. The controlled chemistry of an automated DNA synthesizer allows for the synthesis of longer, higher quality DNA molecules than is possible with the first method. Also, the nucleic acid molecules synthesized can be purified prior to the coupling step. The nucleic acids can be attached to the substrate as described in U.S. Pat. No. 5,837,860.

Thus, for example, covalently immobilized nucleic acid molecules may be used to detect specific PCR products by hybridization where the capture probe is immobilized on the solid phase or substrate (Ranki, 1983, Gene, 21: 77-85; Keller, 1991, Clin. Microbiol., 29: 638-41; Urdea, 1987, Gene, 61: 253-64). A preferred method would be to prepare a single-stranded PCR product before hybridization. A patient sample that is suspected to contain the biomarker molecule, or an amplification product thereof, would then be exposed to the solid-surface and permitted to hybridize to the bound oligonucleotide.

The methods of the present invention do not require that the target nucleic acid contain only one of its natural two strands. Thus, the methods of the present invention may be practiced on either double-stranded DNA (dsDNA), or on single-stranded DNA (ssDNA) obtained by, for example, alkali treatment of native DNA. The presence of the unused (non-template) strand does not affect the reaction.

Where desired, however, any of a variety of methods can be used to eliminate one of the two natural stands of the target DNA molecule from the reaction. Single-stranded DNA molecules may be produced using the ssDNA bacteriophage, M13 (Messing, 1983, Meth. Enzymol., 101: 20-78; see also, Sambrook, 2001, Molecular Cloning: A Laboratory Manuel, 3^{rd} ed. (Cold Spring Harbor Laboratory Press, Cold Spring Harbor, N.Y.).

Several alternative methods can be used to generate single-stranded DNA molecules. For example, Gyllensten, 1988, Proc. Natl. Acad. Sci. U.S.A., 85: 7652-6 and Mihovilovic, 1989, BioTechiques, 7: 14-6 describe a method, termed “asymmetric PCR,” in which the standard “PCR” method is conducted using primers that are present in different molar concentrations.

Other methods have also exploited the nuclease resistant properties of phosphorothioate derivatives in order to generate single-stranded DNA molecules (U.S. Pat. No. 4,521,509; Sayers, 1988, Nucl. Acids Res., 16: 791-802; Eckstein, 1976, Biochemistry 15: 1685-91; Ott, 1987, Biochemistry 26: 8237-41; see also, Sambrook, 2001, Molecular Cloning: A Laboratory Manuel, 3^{rd} ed. (Cold Spring Harbor Laboratory Press, Cold Spring Harbor, N.Y.).

Screening for multiple genes in samples of genomic material according to the methods of the present invention, is generally carried out using arrays of oligonucleotide probes. These arrays may generally be “tiled” for a large number of specific genes. By “tiling” is generally meant the synthesis of a defined set of oligonucleotide probes which is made up of a sequence complementary to the target sequence of interest, as well as pre-selected variations of that sequence, e.g., substitution of one or more given positions with one or more members of the basic set of monomers. i.e. nucleotides. Tiling strategies are discussed in detail in Published PCT Application No. WO 95/11995, incorporated herein by reference in its entirety for all purposes. By “target sequence” is meant a sequence which has been identified as encoding a biomarker of interest or portion thereof, a related polymorphism or mutation (e.g., a single-base polymorphism also referred to as a “biallelic base”) of one of the identified biomarkers. It will be understood that the term “target sequence” is intended to encompass the various forms present in a particular sample of genomic material, i.e., both alleles in a diploid genome.

In a particular aspect, arrays are tiled for a number of specific, identified biomarker sequences. In particular, the array is tiled to include a number of detection blocks, each detection block being specific for a particular biomarker or set of biomarkers. For example, a detection block may be tiled to include a number of probes which span the sequence segment that includes a specific biomarker or a polymorphism thereof. To ensure probes that are complementary to each variant, the probes are synthesized in pairs differing, for example, at the biallelic base.

In addition to the probes differing at the biallelic bases, monosubstituted probes can be generally tiled within the detection block. These monosubstituted probes have up to a certain number of bases in either direction from the polymorphisms, substituted with the remaining nucleotides (selected from A, T, G, C or U). Typically, the probes in a tiled detection block will include substitutions of the sequence positions up to and including those that are 5 bases away from the base that corresponds to the polymorphism. Preferably, bases up to and including those in positions 2 bases from the polymorphism will be substituted. The monosubstituted probes provide internal controls for the tiled array, to distinguish actual hybridization from artifactual cross-hybridization.

A variety of tiling configurations may also be employed to ensure optimal discrimination of perfectly hybridizing probes. For example, a detection block may be tiled to provide probes having optimal hybridization intensities with minimal cross-hybridization. For example, where a sequence downstream from a polymorphic base is G C rich, it could potentially give rise to a higher level of cross-hybridization or “noise,” when analyzed. Accordingly, one can tile the detection block to take advantage of more of the upstream sequence.

Optimal tiling configurations may be determined for any particular biomarker or polymorphism by comparative analysis. For example, triplet or larger detection blocks may be readily employed to select such optimal tiling strategies.

Additionally, arrays will generally be tiled to provide for ease of reading and analysis. For example, the probes tiled within a detection block will generally be arranged so that reading across a detection block the probes are tiled in succession, i.e., progressing along the target sequence one or more nucleotides at a time.

Once an array is appropriately tiled for a given biomarker and related polymorphism or set of polymorphisms, the target nucleic acid is hybridized with the array and scanned. A target nucleic acid sequence, which includes one or more previously identified biomarkers, is amplified by well known amplification techniques, e.g., polymerase chain reaction (PCR). Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single biomarker, in the preferred aspects, the arrays of the invention will include multiple detection blocks, and thus be capable of analyzing multiple, specific biomarkers. For example, preferred arrays will generally include from about 50 to about 4,000 different detection blocks with particularly preferred arrays including from 10 to 3,000 different detection blocks.

In alternate arrangements, it will generally be understood that detection blocks may be grouped within a single array or in multiple, separate arrays so that varying, optimal conditions may be used during the hybridization of the target to the array. For example, it may often be desirable to provide for the detection of those polymorphisms that fall within G C rich stretches of a genomic sequence, separately from those falling in A T rich segments. This allows for the separate optimization of hybridization conditions for each situation.

In one approach, total mRNA isolated from the sample is converted to labeled cRNA and then hybridized to an oligonucleotide array. Each sample is hybridized to a separate array. Relative transcript levels may be calculated by reference to appropriate controls present on the array and in the sample.

### Preparation of Nucleic Acid Probes

Using the sequence information provided herein, the nucleic acids may be synthesized according to a number of standard methods known in the art. Oligonucleotide synthesis, is carried out on commercially available solid phase oligonucleotide synthesis machines or manually synthesized using the solid phase phosphoramidite triester method described by Beaucage, 1981, Tetrahedron Letters, 22: 1859-1862.

Once a nucleic acid encoding a biomarker is synthesized, it may be amplified and/or cloned according to standard methods in order to produce recombinant polypeptides. Molecular cloning techniques to achieve these ends are known in the art. A wide variety of cloning and in vitro amplification methods suitable for the construction of recombinant nucleic acids are known to those skilled in the art.

Examples of techniques sufficient to direct persons of skill through in vitro amplification methods, including the polymerase chain reaction (PCR), the ligase chain reaction (LCR), and other DNA or RNA polymerase-mediated techniques are found in Sambrook, 2001, Molecular Cloning: A Laboratory Manual, 3^{rd} ed. (Cold Spring Harbor Laboratory Press, Cold Spring Harbor, N.Y.

Once the nucleic acid for a biomarker is cloned, a skilled artisan may express the recombinant gene(s) in a variety of engineered cells. Examples of such cells include bacteria, yeast, filamentous fungi, insect (especially employing baculoviral vectors), and mammalian cells. It is expected that those of skill in the art are knowledgeable in the numerous expression systems available for expressing the biomarker proteins of the invention.

### Kits

Kits for practicing the methods of the invention are further provided. By “kit” is intended any manufacture (e.g., a package or a container) comprising at least one reagent, e.g., an antibody, a nucleic acid probe, etc. for specifically detecting the expression of a biomarker of the invention. The kit may be promoted, distributed, or sold as a unit for performing the methods of the present invention. Additionally, the kits may contain a package insert describing the kit and including instructional material for its use.

In a particular embodiment, kits for practicing the immunocytochemistry methods of the invention are provided. Such kits are compatible with both manual and automated ELISA screening. These kits comprise at least one antibody directed to a biomarker of interest immobilized to the surface of a microtitre plate and chemicals for the detection of antibody binding to the biomarker. Any chemicals that detect antigen-antibody binding may be used in the practice of the invention. In some embodiments, the detection chemicals comprise a labeled polymer conjugated to a secondary antibody. For example, a secondary antibody that is conjugated to an enzyme that catalyzes the deposition of a chromogen at the antigen-antibody binding site may be provided. Such enzymes and techniques for using them in the detection of antibody binding are well known in the art. In one embodiment, the kit comprises a secondary antibody that is conjugated to an HRP-labeled polymer. Chromogens compatible with the conjugated enzyme (e.g., DAB in the case of an HRP-labeled secondary antibody) and solutions, such as hydrogen peroxide, for blocking non-specific staining may be further provided.

The kits of the present invention may further comprise a peroxidase blocking reagent (e.g., hydrogen peroxide) and a protein blocking reagent (e.g., purified casein).

Positive and/or negative controls may be included in the kits to validate the activity and correct usage of reagents employed in accordance with the invention. Controls may include samples, such as tissue sections, cells fixed on glass slides, etc., known to be either positive or negative for the presence of the biomarker of interest. The design and use of controls is standard and well within the routine capabilities of those of ordinary skill in the art.

One of skill in the art will further appreciate that any or all steps in the methods of the invention could be implemented by personnel or, alternatively, performed in an automated fashion. Thus, the steps of body sample preparation, sample staining, and detection of biomarker expression may be automated.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### Surfactant Protein-D

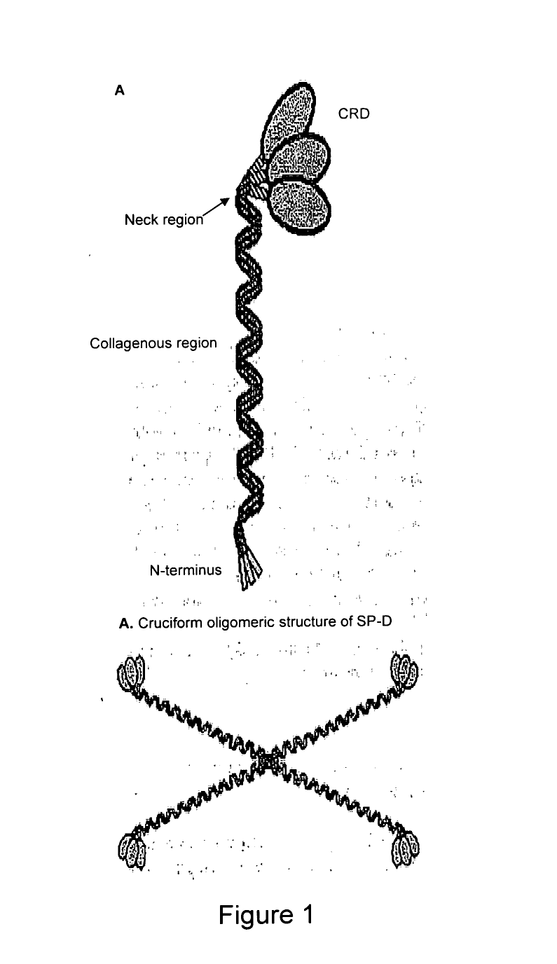

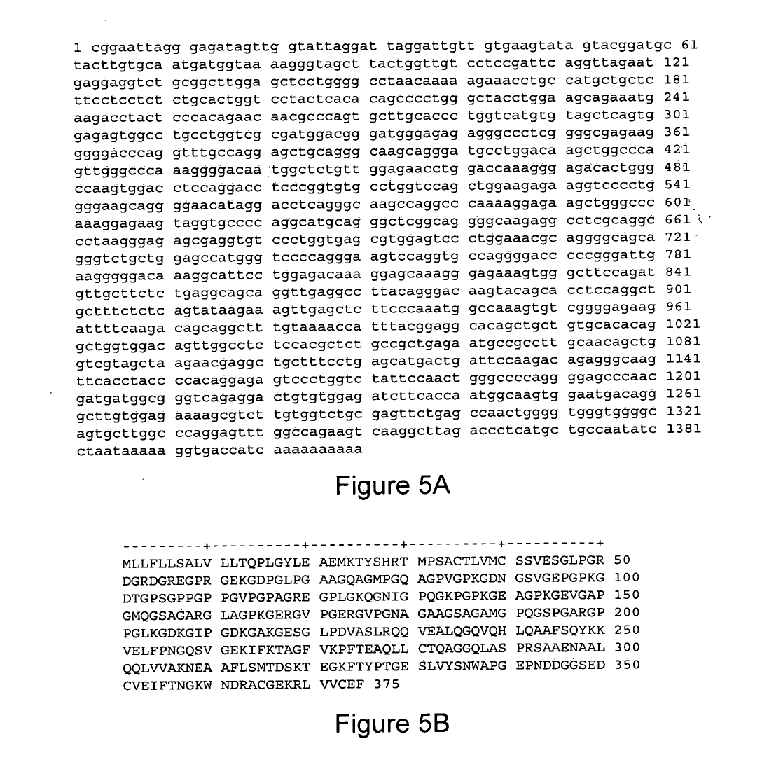

The molecular structure of SP-D is depicted in FIG. 1. The nucleotide sequence (SEQ ID NO.: 1) is depicted in FIG. 5A (Accession No.: X65018). The protein sequence (SEQ ID NO.: 2) is depicted in FIG. 5B (Accession No.: P35247) Collectins are composed of structurally similar C-terminal carbohydrate recognition domains (CRDs) attached to a neck and an N-terminal collagenous region (FIG. 1A). The quaternary structure of collectins formed by higher order oligomers bound through the N-terminally located cysteines forms a “tetrameric cross” with anti-inflammatory properties.

### Human Subjects

To study the role of SP-D in COPD, 20 subjects with varying degrees of COPD (8 former smokers and 12 current smokers) and 15 asymptomatic healthy control subjects (5 never smokers, 3 remote former smokers, and 7 current smokers), were recruited utilizing direct advertising in the Philadelphia area. Volunteers deemed eligible after a preliminary phone screen were scheduled for a screening visit, during which a detailed medical history, tobacco history, medication history, and physical examination were performed. Smoking status was confirmed by urine cotinine levels in all subjects. All subjects underwent spirometry, lung volume assessment by plethysmography, and measurement of the diffusing capacity for carbon monoxide. To qualify for the healthy non-smoker cohort, subjects were required to be asymptomatic non-smokers with a lifetime tobacco exposure of <10 pack years and no tobacco in the last year. In addition, they were required to demonstrate normal pulmonary function at screening. Healthy smokers were required to be current, asymptomatic smokers with a lifetime tobacco exposure of >10 pack years and normal pulmonary function at screening. COPD subjects were required to have a lifetime tobacco exposure of >10 pack years and to demonstrate incompletely reversible airflow obstruction with a post-bronchodilator forced expiratory volume in one second (FEV1)<80% predicted on screening spirometry. Disease severity was established using the Global Initiative for Chronic Obstructive Lung Disease (GOLD) classification (Workshop Report: Global Strategy for the Diagnosis, Management, and Prevention of Chronic Obstructive Pulmonary Disease, 2006 update, www.goldcopd.org). Exclusion criteria included a history of asthma or alpha-1 antitrypsin deficiency, significant medical disease other than COPD (benign conditions such as well-controlled hypertension on minimal medication were allowed), infection in the 3 weeks prior to screening, hospital admission in the 3 months prior to screening, and prior lung resection. Informed consent was obtained from all subjects and the study protocol was approved by the Institutional Review Board of the University of Pennsylvania.

All included subjects underwent bronchoscopy one or more times during the study. At each bronchoscopy, bronchoalveolar lavage fluid (BALF) was collected by sequential instillation of three 50 mL aliquots of sterile saline into a subsegmental bronchus followed by gentle syringe suction. BALF was centrifuged at 1100 rpm for 10 minutes and the cell-free supernatant was separated from the cell pellet and preserved with a protease inhibitor tablet (Roche Diagnostics, from Fisher Scientific, NC9225286) before freezing for subsequent batched analysis.

Of the 35 subjects included in this study, 9 underwent only one bronchoscopy, 22 underwent 2 separate bronchoscopies (each with a separate BALF sample), and 4 underwent 3 bronchoscopies, depending on their participation in prior observational bronchoscopy studies at our institution. Bronchoscopies for any given subject were separated by a minimum of 3 months and no more than 9 months. For each subject, the average of all SP-D levels obtained from the multiple BALF samples was used for all analyses.

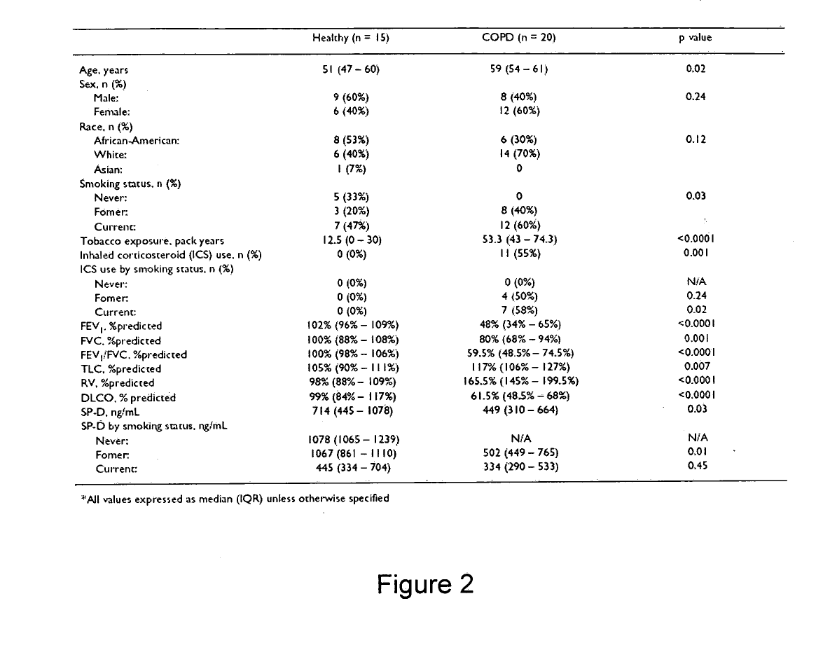

Twenty subjects with varying degrees of COPD (8 former smokers and 12 current smokers) and 15 asymptomatic healthy control subjects (5 never smokers, 3 remote former smokers, and 7 current smokers). Subject characteristics by disease status are listed in FIG. 2. Among subjects with COPD, 9 were classified by the Global Initiative for Chronic Obstructive Lung Disease (GOLD) criteria as GOLD 2 in severity, 8 were classified as GOLD 3, and 3 were classified as GOLD 4. Although GOLD 4 subjects had lower SP-D levels than GOLD 2 and 3 subjects (median SPD levels 201 ng/mL for GOLD 4 and 461 ng/mL for GOLD 2 and 3), this difference did not reach statistical significance (p=0.37). As a result, all GOLD stages were pooled together for further analyses. Healthy control subjects were significantly younger than COPD subjects (median ages 51 and 59, respectively, p=0.02). Although not statistically significant, there were also differences in both gender and race between healthy controls and COPD subjects. Therefore, we adjusted for each of these differences in the primary analysis. As shown in FIG. 2, lung function parameters were lower while tobacco exposure was greater among those with COPD relative to healthy controls.

Previously published studies on BALF SP-D levels from a wide range of laboratories have shown only single sample measurements. To investigate the variability in individual BALF SP-D levels over time, we compared SP-D levels in repeated BALF samples from subjects who underwent multiple bronchoscopies as described in the Methods. One-way analysis of variance revealed marked within subject variability over time (standard deviation 215 ng/ml) in BALF SP-D levels from repeat samples obtained within 3-9 months. This value was nevertheless lower than the between-subject variability (standard deviation 306 ng/ml). To attain the most representative measure for further analyses we used the average of SP-D levels obtained from each subject.

### Human SP-D ELISA

Levels of SP-D and Clara cell protein (CCP-16, an anti-inflammatory protein secreted by Clara cells) were measured in the cell free supernatant of BALF using an Enzyme-Linked ImmunoSorbent Assay (ELISA) kit (Biovendor Inc, Brno, Czech Republic). ELISA was performed according to the manufacturer's instructions. Total protein was assessed by standard Bradford assay. Given that the ELISA measures absolute SP-D or CCP-16 concentrations we are presenting the levels without normalization for total protein content of the BAL. SP-D levels normalized to the optical density obtained for protein measurements did not significantly alter the results.

### Alveolar Type II Cell Culture

Cells were isolated from adult rat lungs digested by collagenase I and collagenase IA (Sigma, St. Louis, Mo.) as previously described (Kaczku, 2006, Pharmacol. Ther. 110:14-34; Bates et al., 2002, Am. J. Physiol. Lung Cell. Mol. Physiol. 282:L267-76; Cao et al., 2004, J. Allergy Clin. Immunol. 113:439-444). Briefly, non adherent cells were plated in 35 mm plastic culture dishes (2−2.5×10⁶ cells/ml) in Waymouth's MB 752/1 Medium (Invitrogen, Carlsbad, Calif.) with 10% fetal bovine serum and incubated at 37° C., 5% CO₂ overnight (day 0). Approximately 40-60% of all the cells plated were type II cells. This proportion remained constant throughout the 4-day culture period. Twenty-four (24) hours later (day 1) cells were washed and further incubated in serum-free Waymouth's medium with 8-Br-cAMP (100 μM) and isobutylmethylxanthine (100 μM) with or without dexamethasone (10 nM) from Sigma Chemical Co. (St. Louis, Mo.)] for an additional 3 days. Cells were re-fed with fresh medium on day 3 and were harvested on day 0, and day 4. Three individual experiments were performed, each using duplicate cultures.

### Western Blot and Northern Blot Analysis

To detect intracellular surfactant proteins, the cell pellet was sonicated and the supernatant was concentrated by evaporation. Western blot analysis was performed as previously described (Hacsku et al., 2001, Am. J. Respir. Cell. Mol. Bio. 25:45-50). Membranes were incubated with rabbit polyclonal anti-SP-D (1:10,000) or with rabbit polyclonal anti-SP-A (1:7,500).

The 386-nucleotide rat cDNA probe for SP-D was newly prepared using Reverse Transcription-Polymerase Chain Reaction (RT-PCR) and the Retroscript Kit (Ambion, Austin, Tex.). Total RNA was isolated from type II cells by RNeasy kit (Qiagen, Valencia, Calif.) and Northern blot analysis was performed as previously described (Hacsku et al., 2001, Am. J. Respir. Cell. Mol. Bio. 25:45-50).

### Statistical Analysis

In univariate analysis, SP-D levels were tested for association with age, sex, race, disease status, smoking status, total smoke exposure in pack-years, years since quit smoking, and inhaled corticosteroid use. The Wilcoxon's ranksum test or Kruskal-Wallis test were used for categorical variables with two or more than two levels, respectively, and the Spearman test for correlation was used for continuous variables. The association between disease status and SP-D level in BALF was stratified by smoking status to control for possible confounding. Among former smokers, SP-D levels approximated a normal distribution; as a result, multivariable linear regression was used to test whether the association between disease status and SP-D levels within this group was independent of the effect of other measured variables. Covariates were considered confounders if they altered the association between disease and SP-D levels by 15% or more.

Further analyses included a non-parametric test for trend (Cuzick, 1985, Stat. Med. 4:87-90) for SP-D levels among subjects categorized by the combination of smoking and disease status, and a linear regression for SP-D levels among all subjects with smoking status included as a categorical covariate rather than as a restricting factor. Bootstrap resampling was conducted to assess the stability of our observed associations among 1000 repeated random samples of our dataset, using the “bootstrap” command in STATA. This method creates numerous datasets of equal size by random sampling of observations in the original dataset with replacement and then recursively conducts the specified statistical test on each new random sample. Confidence intervals were then identified by the percentile method (i.e. selecting the 2.5 and 97.5 percentile of the distribution of observed coefficients). Lastly, one-way analysis of variance was used to calculate the within-subject variability in SP-D level for subjects with multiple samples available. All analyses were conducted using STATA v.8 (StataCorp LP, College Station, Tex.).

The results of the experiments presented in this Example are now described.

### Example 1

### Cigarette Smoking is Associated with Decreased Levels of SP-D in Bronchoalveolar Lavage Fluid (BALF)

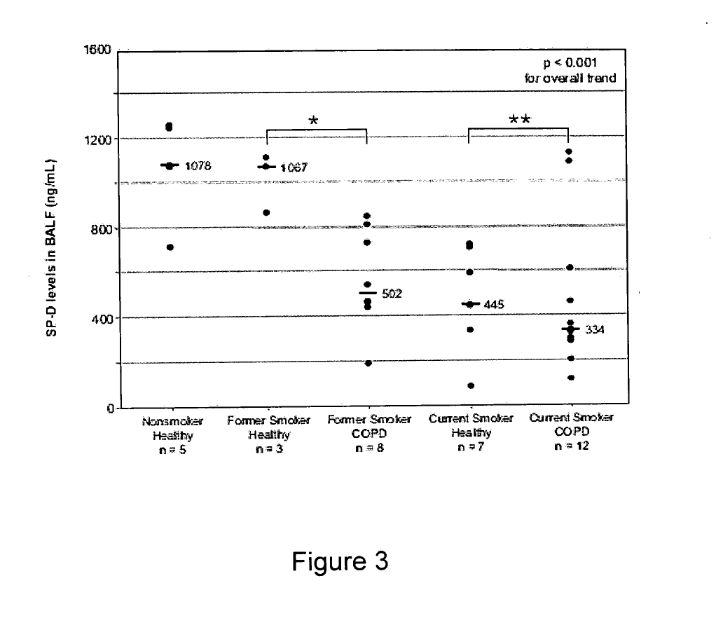

Smoking status had a substantial impact on SP-D levels, with former smokers having levels intermediate between never smokers and current smokers (median SP-D levels 1078 ng/mL, 724 ng/mL, and 363 ng/mL for never, former, and current smokers, respectively (FIG. 3), p=0.04 for the difference between former and never smokers, p=0.004 for the difference between current and never smokers). Importantly, all former smokers in this cohort had quit a minimum of 1.5 years prior to bronchoscopy, eliminating the possibility that the lower levels in former smokers were due to bias from recent cessation.

### Example 2

### COPD is Independently Associated with Pulmonary SP-D Levels

Overall, subjects with COPD had lower SP-D levels in their BALF compared with healthy subjects (median SP-D levels 449 ng/mL and 714 ng/mL, respectively, p=0.03). In order to determine if the effect of disease status was independent of the effect of smoking status, subjects were stratified by smoking status and the effect of disease status among former smokers and current smokers was separately re-examined. Among current smokers, those with COPD had marginally lower SP-D levels in their BALF relative to healthy controls, but this difference was not statistically significant (p=0.45). However, among former smokers, COPD subjects had significantly lower SP-D levels in their +BALF relative to healthy controls (p=0.01). This suggests that COPD is associated with lower levels of SP-D the lung independent of smoking status (FIG. 3).

To further investigate the relationship between smoking and COPD with regard to SP-D levels, the cohort of patients were recategorized into 5 groups consisting of healthy never smokers (n=5), healthy former smokers (n=3), former smokers with COPD (n=8), healthy current smokers (n=7), and current smokers with COPD (n=12). Pulmonary SP-D levels were then analyzed by this new categorization and found that there was a very strong pattern of progressive decline in SP-D levels as determined by a non-parametric test for trend. Median SP-D levels were 108.5 mcg/ml, 107.3 mcg/ml, 49.6 mcg/ml, 43.6 mcg/ml, and 32.5 mcg/ml, respectively (p<0.001).

### Example 3

### Inhaled Corticosteriod Use is Independently Associated with Pulmonary SP-D Levels

Among former smokers, those using inhaled corticosteroids (ICS) had higher SP-D levels than those who do not whether they have COPD or not. The use of inhaled corticosteroid use was examined as a potential confounding factor in the data presented herein.

TABLE 1
Multivariable analysis among former smokers.

| Variables included | Model Coefficient | P Value |
|---|---|---|
| B₀ | 1342 | 0.07 |
| COPD (vs. healthy) | −539 | 0.04 |
| ICS use | 398 | 0.046 |
| Age | −10 | 0.34 |
| Sex | 110 | 0.53 |
| Race (African-American | 216 | 0.26 |
| Vs. White) | | |
| Tobacco (pack-years) | 2.0 | 0.52 |

In univariate analysis, among former smokers with COPD, inhaled corticosteroid use (ICS) was associated with a trend toward higher SP-D levels in comparison to those not using ICS (median SP-D levels 765 ng/mL and 463 ng/mL, respectively, p=0.15). ICS use was not associated with increased SP-D levels among current smokers with COPD.

Multivariable linear regression was then used to test whether the association between disease and SP-D levels among former smokers was independent of ICS use. Interestingly, when ICS use was included as a categorical variable in the model with disease status, both maintained an independent association with SP-D levels. Indeed the magnitude of association between disease status and SP-D levels increased. Even when age, sex, race, and tobacco exposure in pack-years were sequentially forced into the model, both disease and ICS use retained independent associations with SP-D levels (Table 1), eliminating the possibility that our observed associations were due to confounding by these factors.

In order to further examine the stability of these findings, bootstrap resampling was employed, creating 1000 replicates for the model including disease status and ICS use. This analysis confirmed that the associations observed for disease status and use of inhaled corticosteroids were robust (model coefficients [95% CI] were −600 [−826, 0.0] and 290 [13, 516], respectively).

### Example 4

### S-Nitrosylation Alters Multimeric Structure of SP-D

The molecular weight of native (untreated) SP-D, a major component of the BALF exceeded 1000 kD, and was barely able to migrate into a gel during electrophoresis. After undergoing in vitro S-nitrosylation by adding L-SNOC to the BALF fluid, the size of the SP-D complex was reduced such that dodecameric, trimeric and monomeric forms of the protein were observed in the BALF. Thus oxidative stress can result in the degradation of multimeric SP-D into the pro-inflamatory monomeric and trimeric forms.

### Example 5

### Effects of Dexamethasone on Type II Alveolar Epithelial Cell SP-D Levels

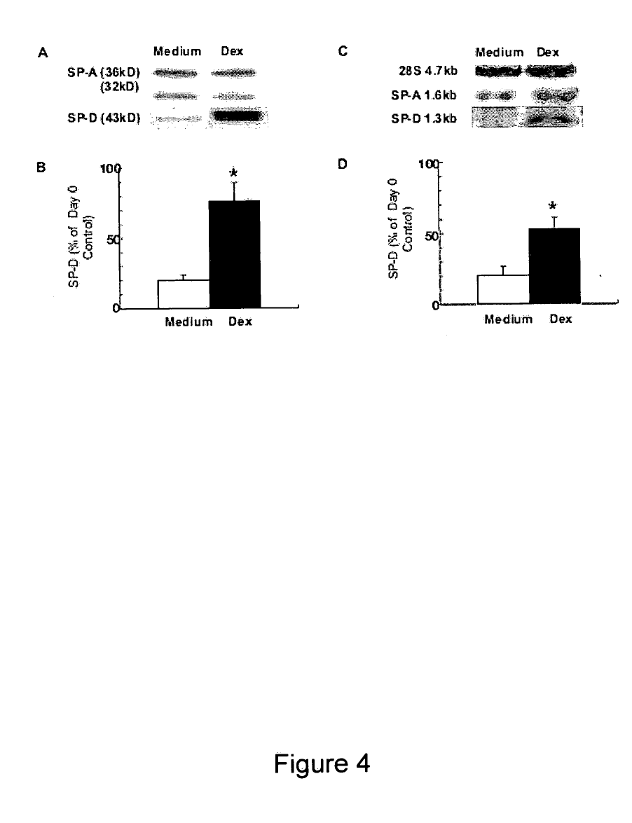

To investigate whether corticosteroids would be capable of directly affecting SP-D synthesis, type II alveolar epithelial cells were isolated from adult rat lungs. Cells were cultured for 4 days in the presence or absence of dexamethasone (10 ng/ml). FIG. 4 demonstrates that dexamethasone induced a significant increase in SP-D expression (p=0.037). This increase was commensurate with a raise in SP-D mRNA in these cells (p=0.041). The effects of dexamethasone were specific because protein expression of the other lung collectin, SP-A did not increase in the presence of this glucocorticoid. These data suggest that dexamethasone is capable of directly enhancing SP-D production by type II alveolar epithelial cells.

### Example 6

### COPD Selectively Affected SP-D Levels in the BALF

To exclude the possibility that lower levels of SP-D among former smokers with COPD were due to a non-specific loss of pulmonary proteins through epithelial destruction, the multivariable model for former smokers was re-examined using SP-D levels normalized to total protein concentration. This model yielded comparable results for the effect of disease on SP-D levels (model coefficient [95% CI]−1162 [−2078, −257], p=0.02) even after controlling for age, race, pack-years of tobacco use, and ICS use (only when sex was included in the model did the coefficient rise to −981 with a p value of 0.079). In addition, Clara cell protein (CCP-16, a protein produced by Clara cells) was analyzed in the BALF in comparison with SP-D levels. Although former smokers with COPD did have a small but statistically significant reduction in CCP-16 levels compared to healthy former smokers (median CCP-16 levels 9.2 mcg/mL vs. 22.6 mcg/mL, respectively, p=0.04), this difference disappeared with control for any potential confounders, including age, race, sex, pack-years of tobacco use, or ICS use. This sharply contrasted with the effect of disease on levels of SP-D, which remained statistically significant despite inclusion of all of these confounders, simultaneously. Thus, the reduced SP-D levels in association with COPD appear to be a specific phenomenon rather than simply reflecting an overall decrease in pulmonary proteins due to loss of airway epithelium.

To further investigate the relationship between smoking and COPD with regard to SP-D levels, the patient cohort was subdivided into 5 groups consisting of healthy never smokers (n=5), healthy former smokers (n=3), former smokers with COPD (n=8), healthy current smokers (n=7), and current smokers with COPD

TABLE 2
Multivariable analysis among all subjects.

| Variables Included | Model Coefficient | P value |
|---|---|---|
| B₀ | 970 | <0.001 |
| Smoking status (former vs. | −356 | 0.02 |
| never) | | |
| Smoking status (current vs. | −628 | <0.001 |
| never) | | |
| Race (African-American | 248 | 0.01 |
| vs. White) | | |
| Race (Asian vs. White) | 103 | 0.71 |

(n=12). We then analyzed pulmonary SP-D levels by this categorization and found that there was a very strong pattern of progressive decline in SP-D levels as determined by a non-parametric test for trend (p<0.001). These data support our observation that both smoking status and COPD have independent effects on SP-D levels. This analysis also highlighted the effect of smoking among healthy subjects: healthy current smokers had lower levels of SP-D in the BALF relative to healthy never smokers (median SP-D levels 445 ng/mL and 1078 ng/mL, respectively, p=0.007) but there was no difference in SP-D levels between healthy former smokers and healthy never smokers (median SP-D levels 1067 ng/mL and 1078 ng/mL, respectively, p=0.65).

In order to determine if the association between COPD and SP-D levels would remain despite including all smoking strata, the multivariable linear regression was repeated on the entire cohort (Table 2), including smoking status as a categorical covariate. Due to probable collinearity, it was not possible to develop a stable model including an interaction term between disease status and smoking status. When both variables were included in the model as main effects without an interaction, the association of smoking status with SP-D levels was stronger than that of disease status with SP-D levels. When race was added to the model with smoking status (omitting disease status), the estimates of association for smoking strata were not altered. However, African-American race was associated with significantly higher SP-D levels compared to white race.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of identifying an individual having lung disease that is responsive to treatment for said lung disease, said method comprising measuring the level of a biomarker in a body sample obtained from said individual, wherein when the level of said biomarker in said sample is greater than the level of the same biomarker in an otherwise identical sample obtained from the same individual prior to commencement of treatment, this is an indication that said individual is responsive to said treatment.

2. The method of claim 1, wherein said individual is a mammal selected from the group consisting of a mouse, a rat, a non-human primate, and a human.

3. The method of claim 2, wherein said mammal is a human.

4. The method of claim 1, wherein said lung disease is COPD.

5. The method of claim 1, wherein said lung disease is asthma.

6. The method of claim 1, wherein said biomarker is a surfactant protein.

7. The method of claim 6, wherein said surfactant protein is surfactant protein D.

8. The method of claim 1, wherein said treatment is administration of a corticosteroid.

9. The method of claim 8, wherein said treatment is inhalation of a corticosteroid.

10. The method of claim 1, wherein said body sample is selected from the group consisting of a tissue, a cell and a bodily fluid.

11. The method of claim 10, wherein said bodily fluid comprises pulmonary sputum and/or bronchoalveolar lavage fluid.

12. The method of claim 1, wherein said measuring of said biomarker comprises an immunoassay for assessing the level of said biomarker in said sample.

13. The method of claim 12, wherein said immunoassay is selected from the group consisting of Western blot, ELISA, immunoprecipitation, immunohistochemistry, immunofluorescence, radioimmunoassay, dot blotting, and FACS.

14. The method of claim 1, wherein said measuring of said biomarker comprises a nucleic acid assay for assessing the level of a nucleic acid encoding said biomarker in said sample.

15. The method of claim 14, wherein said nucleic assay is selected from the group consisting of a Northern blot, Southern blot, in situ hybridization, a PCR assay, and an RT-PCR assay.

16. A method of assessing the efficacy of a treatment for lung disease in an individual, said method comprising obtaining a first sample from said individual having said lung disease and measuring the level of SP-D in said first sample, obtaining a second sample from the same individual at a time subsequent to obtaining said first sample, wherein said time is subsequent to the onset of treatment, and measuring the level of SP-D in said second sample, wherein when the level of SP-D is higher in said second sample than the level of SP-D in said first sample, said treatment is efficacious.

17. The method of claim 16, wherein said individual is a mammal selected from the group consisting of a mouse, a rat, a non-human primate, and a human.

18. The method of claim 17, wherein said mammal is a human.

19. The method of claim 16, wherein said lung disease is COPD.

20. The method of claim 16, wherein said lung disease is asthma.

21. The method of claim 16, wherein said biomarker is a surfactant protein.

22. The method of claim 21, wherein the surfactant protein is surfactant protein D.

23. The method of claim 16, wherein said treatment of the administration of a corticosteroid.

24. The method of claim 23, wherein said treatment is inhaled corticosteroid.

25. The method of claim 16, wherein said body sample is selected from the group consisting of a tissue, a cell and a bodily fluid.

26. The method of claim 25, wherein said bodily fluid comprises pulmonary sputum or bronchoalveolar lavage fluid.

27. The method of claim 16, wherein said measuring of said biomarker comprises an immunoassay for assessing the level of said biomarker in said sample.

28. The method of claim 16, wherein said measuring of said biomarker comprises a nucleic acid assay for assessing the level of a nucleic acid encoding said biomarker in said sample.

29. A composition comprising a plurality of oligonucleotides attached to a substrate surface, wherein each of said oligonucleotides is a nucleic acid encoding a biomarker or a fragment thereof, or is complementary to said biomarker or said fragment thereof, wherein said biomarker is surfactant protein D.

30. The composition of claim 29, wherein the substrate surface is a membrane, a chip, a bead, a microsphere or a microchip.

31. A composition comprising a plurality of peptides attached to a substrate surface, wherein each of said peptides is a biomarker or a fragment thereof, wherein said biomarker is surfactant protein D.

32. The composition of claim 31, where the substrate surface is a membrane, a chip, a bead, a microsphere or a microchip.

33. A composition comprising a plurality of antibodies attached to a substrate surface wherein said antibody specifically binds a biomarker or a fragment thereof, wherein said biomarker is surfactant protein D.

34. The composition of claim 33, where the substrate surface is a plate, a membrane, a solid support, a chip, a bead, a microsphere or a microchip.

35. The antibody of claim 33, wherein said antibody comprises a detectable label.

36. The antibody of claim 35, wherein said detectable label is selected from the group consisting of a radioactive, a fluorescent, a biological, and an enzymatic label.

37. A kit comprising a composition for measuring the level of a biomarker in a body sample, said kit comprising at least one antibody that specifically binds said biomarker or a fragment thereof, said kit comprising instructional material for the use thereof.

38. The kit of claim 37, wherein said mammal is a human.

39. The kit of claim 38, wherein said human has been diagnosed with COPD.

40. The kit of claim 38, wherein said human has been diagnosed with asthma.

41. The kit of claim 37, wherein said composition comprises at least one antibody that specifically binds a biomarker or a fragment thereof, wherein said biomarker is surfactant protein D.

42. The kit of claim 37, wherein at least one of said antibodies is bound to a substrate surface.

43. The kit of claim 37, wherein said antibody comprises a detectable label.

44. The kit of claim 43, wherein said detectable label is selected from the group consisting of a radioactive, a fluorescent, a biological, and an enzymatic label.

45. A kit comprising a composition for measuring the level of a biomarker in a body sample, said kit comprising at least one nucleic acid, wherein said nucleic acid encodes said biomarker or a fragment thereof, or is complementary to said biomarker or a fragment thereof, said kit further comprising an instructional material for the use thereof.

46. The kit of claim 45, wherein said mammal is a human.

47. The kit of claim 46, wherein said human has been diagnosed with COPD.

48. The kit of claim 46, wherein said human has been diagnosed with asthma.

49. The kit of claim 45, wherein said biomarker is surfactant protein D.

50. The kit of claim 45, wherein said nucleic acid probe is immobilized on a solid support.

51. The kit of claim 50, wherein said nucleic acid probe is linked to a detectable label.

52. The kit of claim 51, wherein said label is selected from a radioactive, a fluorescent, a biological and an enzymatic label.
